(19) United States
(12) Patent Application Publication
(10) Pub. No.: US 2021/0290417 A1
(43) Pub. Date: Sep. 23, 2021

(54) MULTI LUMEN RELEASE DEVICE

(71) Applicants: Maria Cecilia Braga Braile, Sao Jose do Rio Preto/SP (BR); BRAILE BIOMEDICA INDUSTRIA COMERCIO E REPRESENTACOES LTDA., Sao Jose do Rio Preto - Sao Paulo (BR)

(72) Inventors: Domingo MARCOLINO BRAILE, Sao Jose do Rio Preto - Sao Paulo (BR); Rafael BRAILE CUNHA, Sao Jose do Rio Preto - Sao Paulo (BR); Wellington BRENO FERNANDES, Sao Jose do Rio Preto - Sao Paulo (BR); Fabio LUIZ COSTA PEREIRA, Vitoria - Espirito Santo (BR); Antonio FIOROTTO JUNIOR, Sao Jose do Rio Preto - Sao Paulo (BR); Ana Luiza PEREIRA DA SILVA, Sao Jose do Rio Preto - Sao Paulo (BR)

(21) Appl. No.: 17/261,143

(22) PCT Filed: Jul. 18, 2019
(86) PCT No.: PCT/BR2019/050286

§ 371 Date: Jan. 18, 2021

Related U.S. Application Data
(60) Provisional application No. 62/699,764, filed on Jul. 18, 2018.

(51) Int. Cl. A61F 2/954; A61F 2/95

(52) U.S. Cl. CPC A61F 2/954; A61F 2/9517

(57) ABSTRACT

The present invention is related a release device that pre-catheterizes the lateral branches in the catheter itself, which has multi channels for passage of each of the wires, preventing the occurrence of strictures in the wires and delivering the already pre-catheterized prosthesis, making the procedure faster and with a lower need for total scopia time. The main advantages achieved by using this device is a decrease in the rate of complications in the release of the prosthesis, shorter surgery time, shorter use of scopia during the procedure, which leads to a lower morbidity of patients and a faster recovery.

### CROSS REFERENCE TO RELATED APPLICATION

This application is a 371 of PCT/BR2019/050286 filed on Jul. 18, 2019 which, in turn, claimed the priority of U.S. Provisional Application No. 62/699,764 filed Jul. 18, 2018, both applications are incorporated herein by reference.

### FIELD OF THE INVENTION

This invention relates generally to accessories for cardiovascular surgery, and more specifically in the minimally invasive devices of endovascular area, for the purpose of transporting endovascular devices, such as stent-grafts, to the target location during procedures for surgical correction of diseases or arterial malformations.

### Fundamentals of the Invention

The arteries are the blood vessels responsible for carrying the blood from the heart to the other parts of the body, they are pulsatile vessels, and the changes in these vessels can lead to decrease of circulation.

Non-treatment of arterial diseases can lead to organ loss, limb loss (amputation) and even death, since rupture of vessels can lead to bulky bleeding. Several comorbidities can affect the blood vessels, including aneurysms, dissections, ruptures and atheromatoses, all of which are amenable to endovascular treatment.

Among these, the most frequent diseases are the aneurysm, which is a dilation, conceptually corresponding to a blood vessel with a diameter 50% larger than normal. The characteristic dilatation of the aneurysm, when it occurs in the aorta that passes through the thorax, is called a thoracic aortic aneurysm, and the dilation that occurs in the segment of the aorta that passes through the abdomen is called the abdominal aortic aneurysm.

Over time dilation may make the artery weak and normal blood pressure may cause it to rupture, with surgical treatment being the only recourse for the repair of an aneurysm. Traditional open surgery had been widely used, and consists of implanting a vascular prosthesis into the aneurysmal sac by means of proximal and distal control. This technique was considered to be gold standard by being well established.

However, in recent years, important contributions have emerged, particularly minimally invasive, a concept in which aortic diseases can be corrected by endovascular prostheses positioned inside the aorta through catheters introduced into the femoral artery under radiological control.

Basically, these advances were introduced by Parodi and contributors in an article published in 1991, in treatments of patients with abdominal aortic aneurysms after extensive experimental studies. The dissection of the descending aorta and aneurysms that worried clinicians and surgeons because of the high mortality and morbidity rates, found in endovascular therapy a very effective option and thus the first works in the literature with encouraging results appeared.

Endoprostheses used in endovascular techniques, also known as stents, are metallic mesh-shaped structures inserted into vascular lumen, whose function is to keep the arterial lumen open, using mechanical pressure. This device avoids restenosis, being self-expanding or expansive balloon type.

In order for these prostheses to be delivered to the site of the disease without large cuts, there is a need to transport it through a device called catheter. The catheters have a small caliber to be compatible with the blood vessels, being flexible, with columnar and bend resistance, besides having a precise positioning system that can be controlled

The structure is composed mainly of PTFE and another polymeric parts, with a gauntlet to perform the release of the stents. The stents are compressed by thin plastic membrane over a small gauge catheter, or are compressed into larger diameter catheters. Thus, catheters are the delivery means for endovascular endoprostheses, bringing the device from the entry site to the implant site.

Currently, when the endoprosthesis implant region has lateral branches that irrigate several organs such as kidneys, liver and intestine, these branches cannot be obstructed, so there is a need to create lateral branches for each of these arteries.

In this case, one by one of the branches needs to be catheterized with a guidewire, requiring a technical skill and a longer period of time for the surgical procedure. In other words, there is an increase in the surgical time, which can harm the patient.

### STATE OF THE ART

Some prior art documents describe solutions for endovascular procedures, with various technologies related to different models of prostheses and methods of delivering these prostheses inside the human body.

Firstly, some solutions about the endovascular stents came out in the 1980s, of which we can highlight the Patent US458058 of Cesare Gianturco, which describes an endovascular stent formed of stainless steel wire of 0.018 inches diameter and arranged in a closed zig-zag pattern. In this case, a flat-ended catheter is used through the sheath to hold the stent in place in the passageway while the sheath is withdrawn from the passageway.

Posteriorly, in the 1990s, Parodi filed a large number of patents for its inventions, such as U.S. Pat. Nos. 5,911,733A and 6,238,432B1, however, the solutions were related to the structure of the stents for specific applications. In U.S. Pat. No. 5,911,733A, for example, an endovascular expander of non-migrant positioning were developed, which includes a tubular body-generally structured by a wire mesh and the expander has a plurality of unidirectional anchorages in proximal arrangement to said tubular body and in the sense of its generatrix. That is, none of the abovementioned documents provide solutions regarding the delivery of endoprosthesis.

For solve the delivery catheter issues there were developed some modality of catheters such as presented in U.S. Pat. Nos. 6,102,942A, 5,824,055A, 5,944,727A, having only the gauntlet, and, in addition, catheters for delivery exclusively in the thoracic region were explored, as in U.S. Pat. No. 6,939,370B2. This last Patent reveals a stent graft deployment device adapted for release of a distal end of a stent graft before the proximal end of the stent graft (25). The arrangement allows movement of at least part of the deployment catheter independently of movement of a proximal end release mechanism has a fixed handle associated with a trigger wire release mechanism and a sliding handle (17) to which the deployment catheter and a capsule (21) are fixed. Although despite promoting a delivery mechanism, there is no possibility of implementing multiple wires, as there is only one channel.

Other catheters are known as multi lumen, however, the application field and mechanisms involved usually diverge from those of the present invention, since a lot of them are dedicated to the dialysis area for transport fluids, as in U.S. Pat. Nos. 8,002,729, 8,496,607, US20090054826A1.

Some of this catheters involves the transport of wires and fluids, as in U.S. Pat. No. 5,250,038, transport of only isolated guide wire, as in U.S. Pat. No. 5,328,480, besides to simple introducers, as in WO2005011788. WO2005011788 describes an apparatus for introducing a first elongate medical device (10) and short wire guide (11) that are coupled together into a work site and remotely disconnecting them within the work site such that a secondary device comprising a catheter member can be introduced over the wire guide to the work site, and/or a second wire guide can be introduced to the work site via a passageway of the primary access device.

The main differences in relation to the present invention is on the guide of a wire, which has a distal end, a proximal end, and a second indicator, wherein the wire guide is movable from a coupled position to an uncoupled. In this solution, there is only a simple introduction of elongate medical devices without the certainty that multiple medical devices would not get involved when introduced concomitantly.

Other patent documents contain catheters multi lumen such as U.S. Pat. No. 8,518,013, WO2006062873, WO2018022404. WO2018022404, for example, displays a Steerable multilumen catheter shaft, comprising a multi lumen catheter shaft body wherein a plurality of longitudinal lumens extends through at least a major length of the catheter shaft body. However, the process of positioning the wires in parallel is conducted in the patient's internal region, with more chances of more likely to cause trauma to the patient.

Another documents which can be cited are the U.S. Pat. Nos. 5,374,245, 5,509,897, 5,797,869, US 2008 0255653 A1, WO 2007 053187, U.S. Pat. No. 88,083,788 B2. This last US Patent is about an apparatus for delivering stents to body lumens include flexible catheter shaft, an expandable member, a tubular prosthesis selectively movable in an axial direction over the expandable member, and a stop member disposed on the catheter shaft near the distal end of the catheter shaft for stopping the prosthesis at a deployment position on the expandable member. The focus of this solution is to guide and positioning prostheses for deployment from a catheter, without establishing a device that could guide multiple wires.

US20110112630 is directed to methods and systems for partial or complete replacement of lumenal valves. An exemplary catheter in accordance with the disclosure includes an elongate body having a proximal end and a distal end, and a retractable sheath mounted on the elongate body proximate the distal end. The sheath and elongate body cooperating to define a first annularly-shaped compartment between the body and sheath. The catheter further includes a valve prosthesis mounted in the compartment, the prosthesis having proximal and distal ends connected to a means for deploying the valve prosthesis from the catheter.

As much as it describes a delivery catheter too, the device to be delivered in US20110112630 is very different from an endoprosthesis, which makes the object of the present invention distinct from the said patent application in some respects.

FIG. 3 of US20110112630 shows some similarity when seen in isolation, however its function is different from that presented for the present invention, because even the device has 5 holes, in only one of them (element 180) there is the passage of a guide wire, and the other 4 holes are used for connection elements (elements 140, 150, 240 and 250) with the function of connecting the prosthesis to the catheter and arching it during implantation, connecting to valve.

In the present invention, the channels give way to wires that do not bind to the endoprosthesis, they only pre-catheterize their lateral branches previously, having their ends free and not acting as a connection between the endoprosthesis and the catheter, preventing the occurrence of strictures in the wires.

US2011/0112630 further recites that: “In accordance with one embodiment, a guidewire lumen 118 is provided along substantially the entire length of elongate body 110 as with typical over the wire (“OTW”) catheters. In accordance with another embodiment, a guidewire lumen 118 is provided only proximate the distal region of elongate body 110 to permit use of catheter 100 as a rapid exchange (“RX”) catheter.”

In contrast, the present invention allows to several wires to be inserted into the different channels of the device, since comprises a tube of polymeric material with one or more channels which run through the entire length and for each channel pass one wire in parallel to the others. This multi-channel configuration is not shown in the abovementioned US application, where only one hole is made for the passage of the guidewire.

In FIG. 4 (b) of US2011/0112630 it is possible to see the connection of the linkages (140) (150) to the valve, differently from what is proposed in this invention to which there is only the passage of guide wires through the entire length of the release device.

Paragraph [056] from US2011/0112630 recites that “a variety of procedures may be accomplished using the teachings herein. For example, a catheter made in accordance with the teachings relating to FIG. 2 herein to perform partial valve replacements at adjacent valves in a patient's vein, such as for treating leg edema. Accordingly, the valve prostheses can be provided in different sizes to allow for the reduction in size in sequential venous valves to permit one catheter to be used to make two valve replacements”. This corroborates the fact that the application specifications apply only to catheters for valve delivery and not to other functions, such as endoprosthesis delivery.

### BRIEF SUMMARY OF THE INVENTION

The present invention is related a release device that pre-catheterizes the lateral branches in the catheter itself, which has multi channels for passage of each of the wires, preventing the occurrence of strictures in the wires and delivering the already pre-catheterized prosthesis, making the procedure faster and with a lower need for total scopia time. The main advantages achieved by using this device is a decrease in the rate of complications in the release of the prosthesis, shorter surgery time, shorter use of scopia during the procedure, which leads to a lower morbidity of patients and a faster recovery.

### BRIEF DESCRIPTION OF THE DRAWINGS

FIG. 1 is a front view of the Multi lumen release device 1 with the channels 2.

FIG. 2 is a side view of the Multi lumen release device 1 with the channels 2 which.

FIG. 3 is an exploded view of the catheter with the device 1.

### DETAILED DESCRIPTION OF THE INVENTION

The present invention is a device, which is a tube made of polymeric material, which comprises one or more channels, whose function is to guarantee the passage of the wires without crossing, in surgical procedures which involves the transportation of an endoprosthesis and branches adjacent to it.

Using this device is possible to pre-catheterize the wires, only requiring the release of each of them, without the need to capture each of the wires independently, reducing surgery time and use of the scopia.

The device is applied together with a catheter, which brings the possibility of implanting more complex prostheses with greater ease in its application.

Such device may have its femoral or brachial access and is introduced to the other end, creating a connection for the wires to pass in parallel.

One or more wires are added in the conventional catheter, and such wires pass through the respective prosthesis channels and later into the multi lumen catheter. This ensures that all the wires of the connections pass through the patient's body without using the scopia or the capture of the wires within the patient.

With all this process done outside the patient, it becomes more precise and less tiring for medical staff, and also eliminating a considerable time of exposure to the scopia.

The device is withdrawn via brachial as the prosthesis catheter enters the patient via femoral, and when the prosthesis arrives in the proper position, then it is released with all the connection paths already pre-established by the wires, with the assurance that they did not meet each other on the way.

In the next steps each wire is selected in the brachial access to insert its respective complement. At this stage, the wire is already guiding the complement into the prosthesis connection, and the medical team will only need to catheterize the patient's artery. Eliminating the time to catheterize the prosthesis connection, the medical staff gains not only time but also avoids the stress of this step.

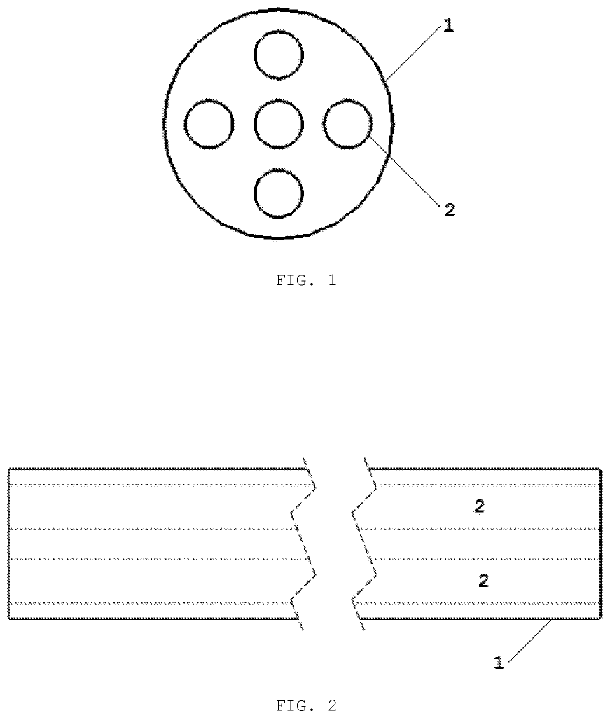

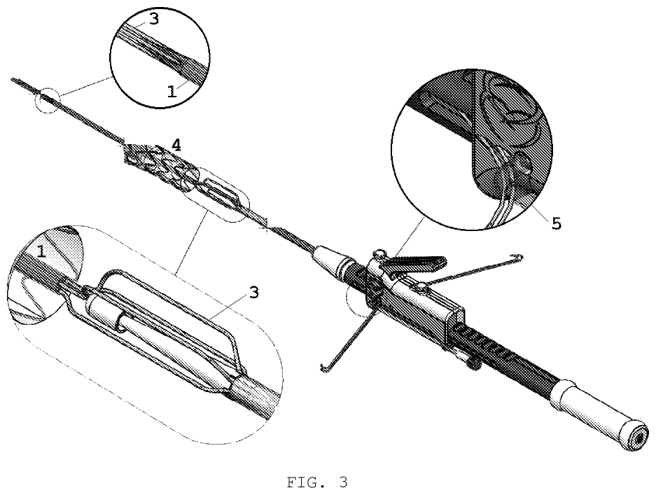

The multi lumen release device 1 is a tube made of polymeric material, which comprises one or more channels 2, and in each channel pass one wire 3, preventing the occurrence of strictures in the wires and delivering the already pre-catheterized prosthesis, according to FIG. 1.

The channels 2 run through the entire length of the device 1, according to FIG. 2. The polymeric tube has an elongate conformation, compatible with the vessel of the human body.

The polymeric tube may be lengthwise movable in the catheter where it is inserted and the wire is inserted inside the catheter and inside the prosthesis branch.

Each wire 3 coming from the catheterization equipment 5 where the wires pass through the inner part of the catheter and into the branches of the prosthesis, extends longitudinally in direction to the multi lumen release device 1, wherein the device is involved by the stent 4.

The catheter is inserted at one end of the human body, such as, but not limited to, the leg, and the branches coming out of the other end of the human body, such as, but not limited to, the arm, using the organism ducts as a channel.

1. A multi lumen release device for delivering endoprosthesis comprising a tube of polymeric material with one or more channels which run through an entire length and for each channel pass one wire in parallel to the others,
wherein said device is inserted in a multiple lumen catheter;
and wherein wires pre-catheterize lateral branches in the catheter.

2. The device according to claim 1, wherein the catheter is inserted at one end of an extremity of a human body, and the branches come out of an opposite end of another extremity of the human body, using the subject's ducts as a channel.

3. (canceled)

4. The device according to claim 1, wherein the tube of polymeric material has an elongate conformation, compatible with a vessel of the human body.

5. The device according to claim 1, wherein the tube of polymeric material is lengthwise movable in the catheter where it is inserted.

6. The device according to claim 1, wherein the wire is inserted inside the catheter and inside a prosthesis branch.
